(19) United States
(12) Patent Application Publication
(10) Pub. No.: US 2010/0168868 A1
(43) Pub. Date: Jul. 1, 2010

(54) LAYERED GRADIENT MATERIAL FOR BIOLOGICAL USE AND METHOD FOR PRODUCING THE SAME

(75) Inventors: Michiharu Okano, Tokyo (JP); Hiroshi Izui, Tokyo (JP); Yasumasa Fukase, Tokyo (JP); Yasuaki Tokuhashi, Tokyo (JP)

Correspondence Address: BIRCH STEWART KOLASCH & BIRCH, PO BOX 747, FALLS CHURCH, VA, 22040-0747, US

(73) Assignee: Nihon University

(21) Appl. No.: 12/517,032

(22) PCT Filed: Nov. 29, 2007
(86) PCT No.: PCT/JP2007/073040

§ 371 Date: Dec. 30, 2009

(30) Foreign Application Priority Data
Nov. 30, 2006 (JP) 2006-323181

(51) Int. Cl. A61F 2/28; C04B 35/64

(52) U.S. Cl. 623/23.51; 264/642

(57) ABSTRACT

A layered gradient material which comprises a plurality of layers composed of a mixture of hydroxyapatite and calcium tertiary phosphate and is obtained by producing a layered body by stacking a plurality of layers in which the compounding ratio of hydroxyapatite to calcium tertiary phosphate is stepwise or continuously changed and then sintering the layered body by the spark plasma sintering method is excellent in mechanical strength such as compressive strength, flexural strength and Young's modulus, and further, is excellent in biological reactivity such that in the case where it is used as a bone substitute material, bone destruction and new bone reconstruction actively occur. Accordingly, it is extremely useful as a biological material such as a bone substitute material, artificial tooth root and dental cement.

### TECHNICAL FIELD

The present invention relates to a layered gradient material which comprises hydroxyapatite (hereinafter referred to as “HAp” and tribasic calcium phosphate (TCP) and is used as a biological material such as a bone substitute material, artificial tooth root or dental cement, and a method for producing the layered gradient material.

### BACKGROUND ART

In the field of oral-maxilla surgery, loss of bones sometimes occurs due to external wound or surgical operation such as removal of tumors or cysts formed at maxilla bones. Furthermore, in implantation treatment or production of artificial tooth, there are many cases which need increase of alveolar bones, bone transplantation and implantation of bone substitute material. As transplantation materials used for these substitution and reconstruction, mostly fresh self bones are used because bones can be bonded thereto satisfactorily, surely and safely. However, fresh self bones for transplantation have problems that extra surgical invasion is required for gathering transplantation bones in addition to the operation of primary focuses and that there are limitations in supply and shape of bone tissue.

Under the circumstances, development of biological materials to be substituted for self bone transplantation materials has been made. W. R. Brown et al (Patent Document 1) developed self-curing cement comprising calcium phosphate (calcium phosphate cement which is hereinafter referred to as “CPC”), and Fukase et al (Non-Patent Document 1, Non-Patent Document 2, Non-Patent Document 3, Non-Patent Document 4, Non-Patent Document 5, and Non-Patent Document 6) reported that they made improvement in said cement to improve curing reaction and physical properties.

However, said bone substitute material in the form of cement is pasty until curing, and hence it has a degree of freeness in filling, but since strength cannot be assured for a period until curing (24 hours at 38° C.), there is restriction in reinforcement of the site which requires strength, such as body trunk bone. Therefore, block-like bone substitute materials are useful, and conventional dense body or porous body of HAp can ensure strength, but is low in reactivity with bone since it is excellent in biological affinity, and replacement for self bone can hardly be expected. When reactivity with bone is demanded, it is effective to use calcium phosphate such as β-phase TCP (hereinafter referred to as “β-TCP” or “βTCP” which is high in solubility, but β-TCP is inferior to HAp in mechanical strength. Thus, there has been no material which satisfies both strength and reactivity.

Real bones have such features that they comprise dense bone of outer side and have shell structure to exhibit strength and the inner side shows biological reactivity due to bone marrow tissue. Development of artificial materials which satisfy both the mechanical strength and biological reactivity has been desired.

Patent Document 1: U.S. Pat. No. 4,518,430

Non-Patent Document 1: Y. Fukase et al., J. Dent Res 1990; 69(12); 1852-1856

Non-Patent Document 2: Y. Fukase, “Nichidai Shika (Nihon University Dentistry)”, 1990; 64: 190-203

Non-Patent Document 3: M. Ogawa, “Nichidai Shika (Nihon University Dentistry)”, 1995; 69: 561-570

Non-Patent Document 4: H. Uehara, “Nichidai Shika (Nihon University Dentistry)”, 1995; 69: 728-744

Non-Patent Document 5: Y. Fukase et al., J. Oral Sci 1998; 40(2): 71-76

Non-Patent Document 6: S. Wada, “Nichidai Shika (Nihon University Dentistry)”, 1998; 72: 408-420

### DISCLOSURE OF INVENTION

### Problem to be Solved by the Invention

Accordingly, the object of the present invention is to provide an artificial material which have the same mechanical strength and biological reactivity as those of real bones, and a method for producing the same.

### Means for Solving the Problem

In an attempt to solve the above problems, the inventors have considered that according to spark plasma sintering method (SPS method), there can be produced a gradient functional material by changing compounding ratio of a plurality of materials or chemical properties of the materials, and hence there can be produced a material having functions of gradient in composition, biological reactivity and strength changing from the portion comprising 100% of HAp having strength to the portion comprising 100% of β-TCP having biological reactivity in the same specimen by changing the compounding ratio of HAp high in strength, but low in biological reactivity and β-TCP high in biological reactivity, but low in strength. Under the circumstances, they have conducted intensive research on production of gradient materials by SPS method. As a result, the present invention has been accomplished.

Thus, the present invention includes the following inventions (1) to (12).

(1) A layered gradient material for biological use which comprises hydroxyapatite (HAp) and tribasic calcium phosphate (TCP), the compounding ratio of HAp and TCP being changed stepwise or continuously;

(2) a layered gradient material for biological use described in the above (1), wherein TCP is β-phase TCP (β-TCP);

(3) a layered gradient material for biological use described in the above (2), wherein β-TCP has a particle size of 590 μm to 1000 μm;

(4) a layered gradient material for biological use described in any one of the above (1) to (3), wherein HAp has a particle size of 10 μm to 170 μm;

(5) a layered gradient material for biological use described in any one of the above (1) to (4) which is obtained by stacking a plurality of layers in which the compounding ratio of HAp and TCP is stepwise or continuously changed and then sintering the layered body by a pressure sintering method;

(6) a layered gradient material for biological use described in any one of the above (1) to (5) which is used as a bone substitute material;

(7) a method for producing a layered gradient material for biological use which comprises stacking a plurality of layers composed of a mixture of HAp and TCP in which the compounding ratio of HAp and TCP is stepwise or continuously changed and then sintering the resulting layered body by a pressure sintering method;

(8) a method for producing a layered gradient material for biological use described in the above (7), wherein the layered body is sintered by spark plasma sintering method (SPS method);

(9) a method for producing a layered gradient material for biological use described in the above (7) or (8), wherein a plurality of layers composed of a mixture of HAp and TCP in which the compounding ratio of HAp and TCP is stepwise or continuously changed are filled up in a frame and stacked in vertical direction and then the resulting layered body is sintered by SPS method;

(10) a method for producing a layered gradient material for biological use described in any one of the above (7) to (9), wherein TCP is β-TCP;

(11) a method for producing a layered gradient material for biological use described in the above (10), wherein β-TCP has a particle size of 590 μm to 1000 μm;

(12) a method for producing a layered gradient material for biological use described in any one of the above (7) to (11), wherein HAp has a particle size of 10 μm to 170 μm; and

(13) a method for producing a layered gradient material for biological use described in any one of the above (7) to (12) which is used as a bone substitute material.

### ADVANTAGES OF THE INVENTION

The layered gradient material for biological use according to the present invention is excellent in mechanical strength such as compressive strength, bending strength and Young's modulus, and further, is excellent in biological reactivity such that in the case where it is used as a bone substitute material, bone destruction and new bone reconstruction actively occur. Accordingly, it is extremely useful as a biological material such as a bone substitute material, artificial tooth root and dental cement.

### BEST MODE FOR CARRYING OUT THE INVENTION

HAp used in the present invention is a compound represented by Ca₁₀(PO₄)₆(OH)₂ and is a main component constituting a bone. Particularly, HAp has a particle size of preferably 10 μm to 170 μm, more preferably 20 μm to 60 μm. The shape thereof is preferably spheroidal. As preferred examples of HAp, mention may be made of, for example, SHAp-100 (HAp prepared by spheroidizing HAp having the same component as of commercially available HAp-100 which has a particle size of 40 μm and is manufactured by Taihei Kagaku Kogyo Co., Ltd.).

TCP used in the present invention means tribasic calcium phosphate (Ca₃(PO₄)₂), and is similar to HAp in physical properties, solubility and biological affinity. TCP includes high-temperature type α-phase (α-TCP), low-temperature type β-phase (β-TCP) and high-temperature and high-pressure type γ-phase (γ-TCP) which differ in crystal structure, and α-phase TCP and β-phase TCP are largely used as biological materials. α-TCP is high in solubility in water and is converted to HAp upon hydrolysis reaction. Furthermore, β-TCP is also relatively high in solubility in water and is superior in biological affinity, and is largely used for bone filling materials as biologically absorptive implantation materials. In the present invention, there may be used any of α-TCP, β-TCP and β-TCP, and β-TCP is particularly preferred. The particle size of β-TCP is preferably 590 μm to 1000 μm, and β-TCP is preferably in the shape of ground particles.

In the present invention, there may be optionally used apatite hydrogencarbonate, apatite fluoride, titanium, or the like in addition to HAp and TCP.

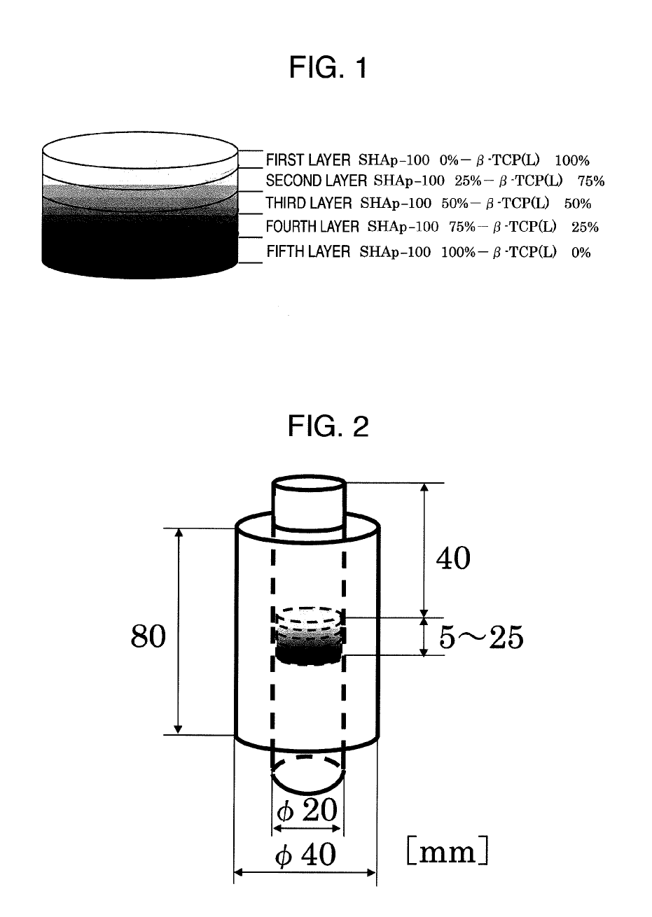

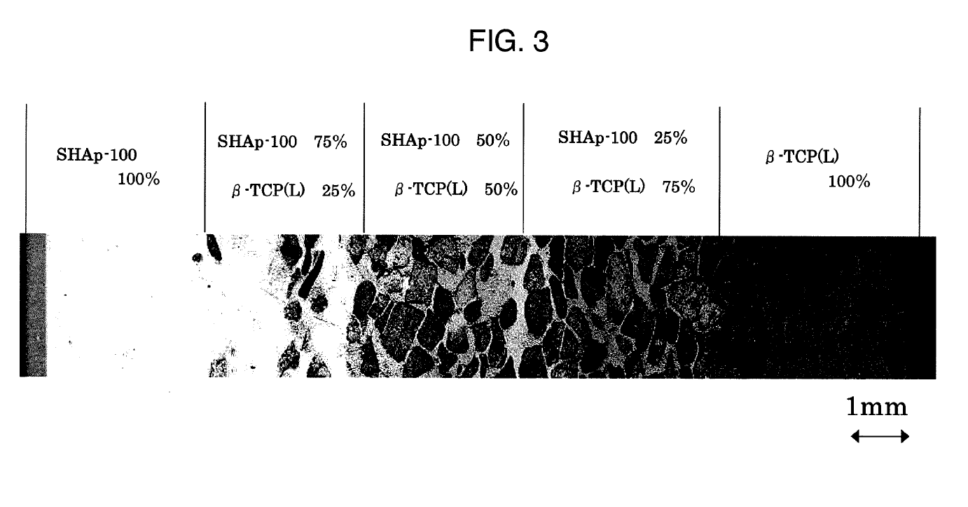

The layered gradient material for biological use of the present invention has such a construction that the compounding ratio of HAp and TCP is stepwise or continuously changed. More specifically, as shown in, for example, FIGS. 1, 5 and 8, mention may be made of gradient materials obtained by pressure sintering a layered body composed of a first layer comprising 100% of β-TCP, a second layer comprising 25% of HAp and 75% of β-TCP in volume ratio, a third layer comprising 50% of HAp and 50% of β-TCP in volume ratio, a fourth layer comprising 75% of HAp and 25% of β-TCP in volume ratio, and a fifth layer comprising 100% of HAp. FIG. 3 shows an SEM photograph of a gradient material obtained by sintering a layered body comprising 5 layers by SPS method. As can be seen from FIG. 3, after sintering the layered body, the resulting gradient body has a compounding ratio of HAp and β-TCP which is nearly continuously changed. As mentioned above, the layered gradient material for biological use according to the present invention has such a construction that the compounding ratio of HAp and TCP in thickness direction is stepwise or continuously changed.

As mentioned above, the gradient material of the present invention is obtained by stacking a plurality of layers composed of a mixture of preferably HAp and TCP in which the compounding ratio of HAp and TCP is stepwise or continuously changed to produce a layered body and then sintering the resulting layered body by a pressure sintering method. The number of the layers stacked is not limited to the above 5 layers, and the layered body may be composed of an optional number of layers, and, for example, it may be a layered body composed of a first layer comprising 100% of TCP and a second layer comprising 100% of HAp, a layered body composed of a first layer comprising 100% of TCP, a second layer comprising 50% of TCP and 50% of HAp in volume ratio and a third layer comprising 100% of HAp, a layered body composed of a first layer comprising 100% of TCP, a second layer comprising 80% of TCP and 20% of HAp in volume ratio, a third layer comprising 60% of TCP and 40% of HAp in volume ratio, a fourth layer comprising 40% of TCP and 60% of HAp in volume ratio, a fifth layer comprising 20% of TCP and 80% of Hap in volume ratio, and a sixth layer comprising 100% of HAp. Preferably, the layered body is composed of about 2-40 layers.

In order to form a layer comprising HAp and TCP, powdery mixtures of HAp and TCP may be mixed well using, if necessary, a pot mill rotating table. Then, layers having the respective compounding ratio are stacked and the resulting layered body is sintered by pressure sintering method. The pressure sintering method includes, for example, hot press (HP) method of carrying out the sintering while pressing the powders, hot hydrostatic pressing (HP) method of carrying out the sintering while omnidirectionally pressing the powders under equal pressure, normal pressure sintering method of carrying out the sintering under normal pressure a molded body previously formed by cold hydrostatic pressing (CIP), or the like, and in the present invention, SPS method (spark plasma sintering method) is preferred. This SPS (spark plasma sintering) method is a technology of synthesis and working of materials of the next generation type capable of performing the sintering in a short time of about 5-20 minutes including heating time and holding time in a region of from low temperature to ultra-high temperature of higher than 2000° C. by directly introducing electric energy in the form of pulse into voids of the pressed powder particles, whereby high energy of high-temperature plasma, namely, spark plasma generated in a moment by spark discharge is effectively applied to heat diffusion and electric field diffusion (Masao Tokita: Journal of Society of Particle Technology, Commentary 30[11] p. 790-804 (1993); New Ceramics, No. 10, p. 43-51, 1997). The spark plasma sintering method is a kind of pressure sintering method using ON-OFF direct current pulse passing method, by which dense sintered bodies can be obtained at a lower temperature and in a shorter time than in conventional hot press (HP) method and hot hydrostatic pressing (HP) method. Furthermore, the spark plasma sintering method is of a direct heat generation system utilizing discharge and Joule's heat at the time of passing great current pulse, and hence is extremely superior in thermal efficiency. Thus, uniform and high quality sintered bodies can be obtained by uniform heating caused by dispersion of the points of the discharge and Joule's heat.

In order to sinter the layered body by SPS method, mixtures of HAp and TCP at the respective compounding ratios are filled in a frame such as die•punch made of high-strength graphite to form layered bodies as shown in FIGS. 2,4 and 7. The shape of the frame may be selected according to the shape of gradient materials to be obtained, and it may be in the form of column, square pillow or the like. The frame is preferably a mold made of high-strength graphite. Then, the apparatus system is allowed to be in vacuous atmosphere or in non-oxidizing atmosphere such as nitrogen gas, argon or the like, and the layered bodies can be sintered by heating with passing pulsed direct current or direct current containing square wave or firstly passing pulsed direct current and then passing direct current containing square wave through the frame containing the layered body. As the spark plasma systems, there are two kinds of systems one of which passes only direct current (available mainly from Sumitomo Coal Mining Co., Ltd.) and another of which passes pulsed direct current for the first period of 0-750 seconds and thereafter passes direct current containing square wave (available mainly from Sodic Co., Ltd.). In the present invention, the spark plasma systems where heating is carried out by passing direct current are preferred.

As the sintering conditions in the present invention, preferred are, for example, a filling pressure in a frame of 5-20 MPa, a heating rate of 50-300° C./min, a sintering temperature of 700-1100° C., a sintering time of 2 minutes or longer, and sintering pressure of 20-75 MPa. After completion of the sintering, the layered body may be cooled at a controlled cooling speed of 80-150° C./min, or may be naturally cooled upon stopping operation of the apparatus. Thus, the layered gradient material for biological use of the present invention can be obtained.

The layered gradient material for biological use of the present invention can be applied to, for example, artificial bones, artificial tooth roots and artificial joints after being formed into suitable shapes depending on uses.

The present invention will be explained in detail below by the following examples, which should not be construed as limiting the invention.

### Example 1

### Production of Layered Gradient Material Comprising HAp and β-TCP

As HAp, there was used SHAp-100 (having a particle size of 40 μm manufactured by Taihei Kagaku Kogyo Co., Ltd.) which was high in mechanical strength, and as β-TCP, there was used β-TCP(L) (β-TCP having a particle size of 590-1000 μm manufactured by Taihei Kagaku Kogyo Co., Ltd.) capable of realizing a porous material into which osteoblasts can enter.

The given amount of powders as shown in Table 1 were mixed using a pot mill rotating table (Model: AN3S manufactured by Nitto Kagaku Co., Ltd.). The volume ratio in Table 1 is assumed to be a volume ratio after sintering.

TABLE 1
Filling amount of each layer

| | | | Filling amount (g) |
|---|---|---|---|
| Layer | Material | Volume ratio | (thickness 20 mm) |
| First layer | β-TCP(L) | 100 | 1.17 |
| Second layer | β-TCP(L) | 75 | 0.87 |
| | SHAp-100 | 25 | 0.28 |
| Third layer | β-TCP(L) | 50 | 0.69 |
| | SHAp-100 | 50 | 0.69 |
| Fourth layer | β-TCP(L) | 25 | 0.33 |
| | SHAp-100 | 75 | 0.97 |
| Fifth layer | SHAp-100 | 100 | 1.12 |

In this Example, the powders were not mixed using a mortar because β-TCP(L) having large particle diameter was not to be ground. The powders were filled in such a manner that those of the layer comprising 100% of β-TCP were filled as the uppermost layer and then those of the following layers were filled in succession as shown in FIG. 1 in a die•punch made of high-strength graphite shown in FIG. 2. After filling, they were sintered under the conditions as shown in Table 2. Sintering was carried out using SUMISEKI spark plasma sintering machine.

TABLE 2
Sintering conditions

| | | |
|---|---|---|
| Filling pressure | 10 | [MPa] |
| Heating rate | 100 | [° C./min] |
| Sintering time | 8 | [min] |
| Sintering temperature | 800 | [° C.] |
| Cooling speed | 100 | [° C./min] |
| Sintering pressure | 22.3 | [MPa] |

The section in longer direction of the specimen after sintered was photographed using SEM and shown in FIG. 3. As can be seen from FIG. 3, there was obtained a gradient material nearly continuously changed in compounding ratio of SHAp-100 and β-TCP(L) by sintering the layered body of five layers by SPS method.

### Example 2

### Evaluation of Mechanical Characteristics of Layered Gradient Material of HAp and β-TCP

(1) Compression Test

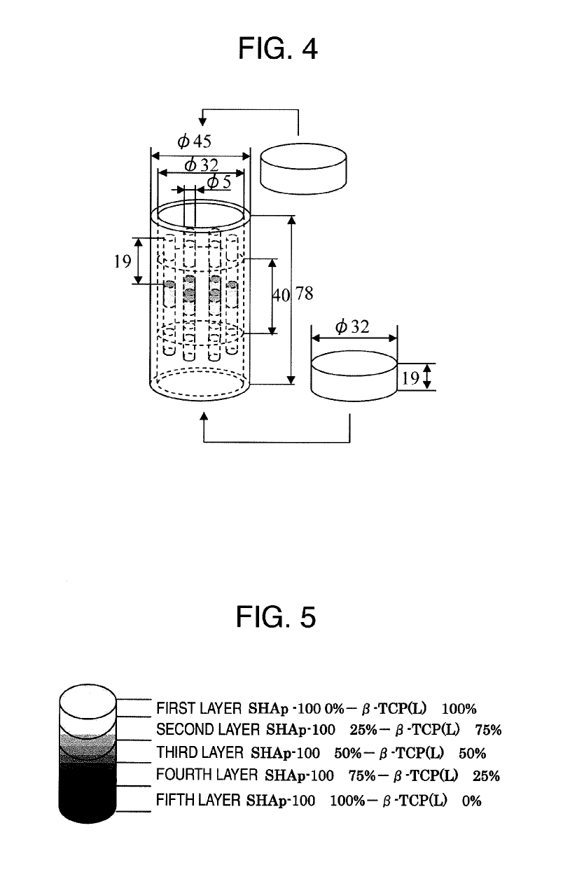

Columnar specimens were prepared using the same SHAp-100 and β-TCP(L) as used in Example 1 using a mold made of high-strength graphite as shown in FIG. 4. As for the shape of the specimens, they had a size of 5 mm in diameter and 12.5 mm in height, and the powders were filled in the mold made of high-strength graphite with adjusting the amounts of the powders as shown in FIG. 5. The sintering conditions employed are shown in Table 3.

TABLE 3
Sintering conditions

| | | |
|---|---|---|
| Filling pressure | 10 | [MPa] |
| Heating rate | 100 | [° C./min] |
| Sintering time | 8 | [min] |
| Sintering temperature | 800 | [° C.] |
| Cooling speed | 100 | [° C./min] |
| Sintering pressure | 66.9 | [MPa] |

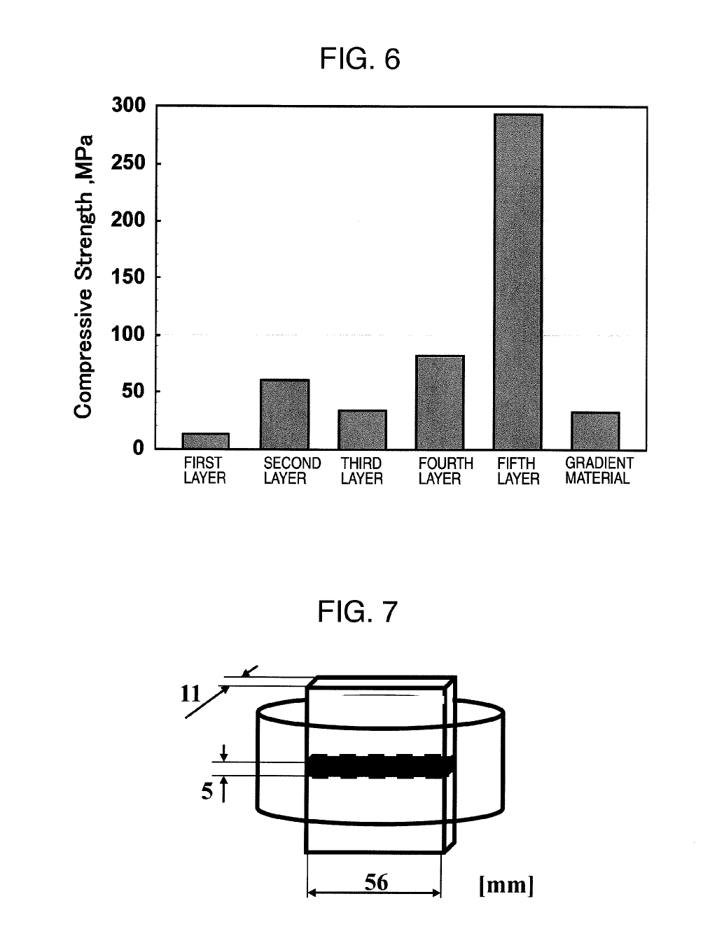

The specimen obtained by sintering was subjected to compression test according to JIS. The results obtained are shown in FIG. 6. In FIG. 6, the compression strengths of the first layer, second layer, third layer, fourth layer and fifth layer are those of the respective sintered layers. As can be seen from the results of FIG. 6, the compression strength of the gradient material was higher than that of the first layer (sintered body comprising only β-TCP(L)), and thus the compression strength was improved in the case of being made into a gradient material.

(2) Bending Strength Test

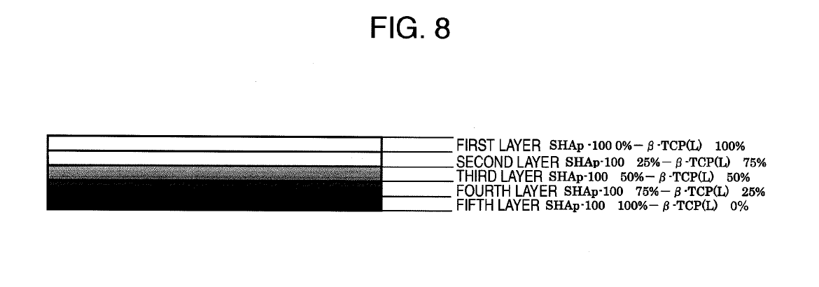

A plate specimen was prepared using the same SHAp-100 and β-TCP(L) as used in Example 1 using a mold made of high-strength graphite shown in FIG. 7. As for the shape of the specimen, it had a size of 11 mm in width, 56 mm in length and 2 mm in thickness, and the respective powders were filled in a mold made of high-strength graphite with adjusting the amounts of the respective powders as shown in FIG. 8. The sintering conditions employed are shown in Table 4.

TABLE 4
Sintering conditions

| | | |
|---|---|---|
| Filling pressure | 10 | [MPa] |
| Heating rate | 100 | [° C./min] |
| Sintering time | 8 | [min] |
| Sintering temperature | 800 | [° C.] |
| Cooling speed | 100 | [° C./min] |
| Sintering pressure | 44.6 | [MPa] |

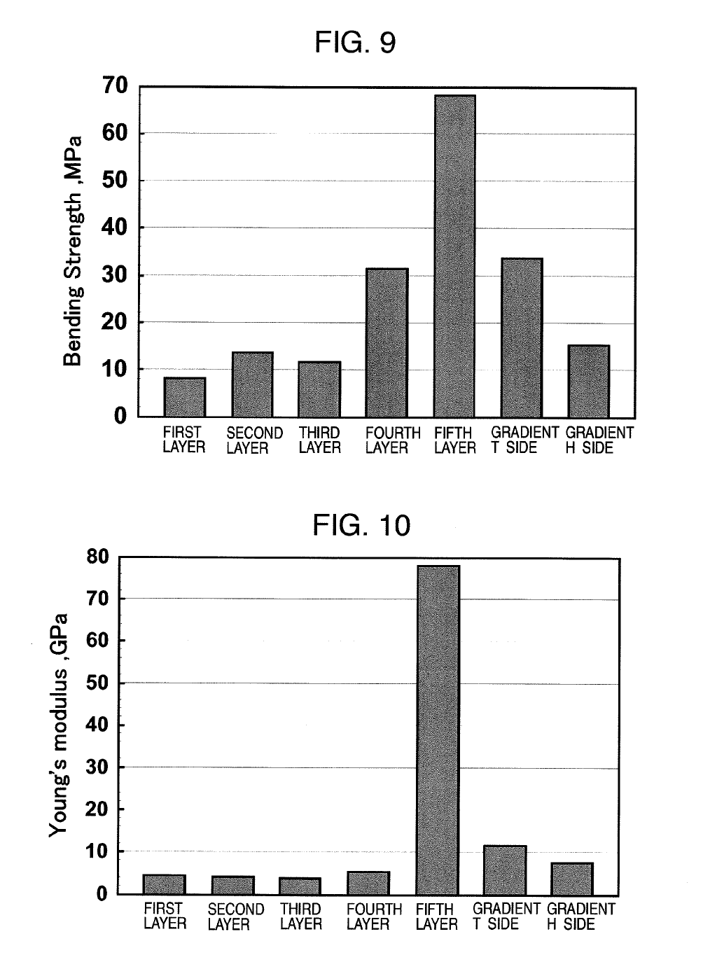

The specimen obtained by sintering was subjected to bending strength test specified in JIS. The results obtained are shown in FIG. 9. In FIG. 9, the bending strengths of the first layer, second layer, third layer, fourth layer and fifth layer are those of the respective sintered layers. Furthermore, in FIG. 9, the gradient T side shows the result of the bending strength test carried out by applying the force to the first layer (the sintered body comprising only β-TCP(L)), and the gradient H side shows the result of the bending strength test carried out by applying the force to the fifth layer (the sintered body comprising only SHAp-100). As can be seen from the results of FIG. 9, the bending strength increased from that of the first layer toward that of the fifth layer except for the third layer as in the case of compression strength. Moreover, with reference to the bending strength of gradient material, the strength of the gradient T side was the same strength as that of the fourth layer. Further, the strength of the gradient H side was nearly twice that of the first layer. Thus, bending strength was improved in the case of being made into gradient material.

(3) Young's Modulus Test

Young's modulus of the specimen used in the bending strength test was measured. The results obtained are shown in FIG. 10. As can be seen from the results of FIG. 10, all of the layers except for the fifth layer (sintered body comprising only SHAp-100) had nearly the same Young's modulus.

### Example 3

### Evaluation of Biological Characteristics of Layered Gradient Material Comprising HAp and β-TCP

(1) Production of Gradient Material

In the same manner as in Example 1, SHAp-100 (having a particle size of 40 μm manufactured by Taihei Kagaku Kogyo Co., Ltd.) was used as HAp, and β-TCP (L) (β-TCP having a particle size of 590-1000 μm manufactured by Taihei Kagaku Kogyo Co., Ltd.) was used as β-TCP, and these powders were filled in a die•punch made of high-strength graphite shown in FIG. 2 in such a manner that those of the layer comprising 100% of β-TCP were filled as the uppermost layer and then those of the following layers were filled in succession as shown in FIG. 1. Then, they were sintered under the conditions shown in Table 5. Sintering was carried out using SUMISEKI spark plasma sintering machine.

TABLE 5
Sintering conditions

| | | |
|---|---|---|
| Filling pressure | 10 | [MPa] |
| Heating rate | 100 | [° C./min] |
| Sintering time | 8 | [min] |
| Sintering temperature | 800 | [° C.] |
| Cooling speed | 100 | [° C./min] |
| Sintering pressure | 44.6 | [MPa] |

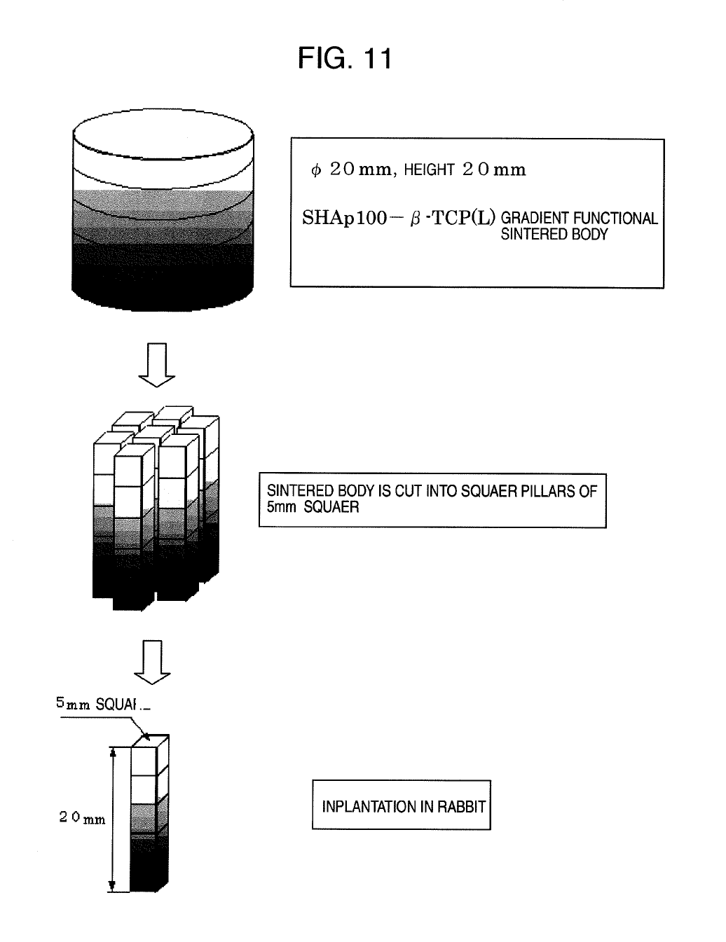

The specimen after sintered was formed into a square pillar of 5×5×20 mm as shown in FIG. 11, which was subjected to the following biological test.

(2) Biological Test

1) Gradient Material

As shown in FIG. 11, a square pillar of 5×5×20 mm was cut out from the sintered body comprising SHAp-100 and β-TCP(L) produced in the above (2) and was subjected to dry air sterilization for implantation in a thighbone of house rabbit.

As a control, a cylindrical SPS sintered body of 20 mm in diameter×20 mm in length comprising 100% of SHAp-100 was produced, and a square pillar of 5×5×20 mm was cut out from the sintered body and was subjected to dry air sterilization for implantation in a thighbone of house rabbit.

2) Experimental Animals

Five female house rabbits (Japanese White having a body weight of about 3.0 kg available from Sankyo Labo Service) which were bred for about two weeks in a feeding test facility were used as experimental animals.

3) Implantation in Bone

The house rabbit was subjected to intravenous injection with pentobarbital sodium (NEMBUTAL manufactured by Sankyo Co., Ltd.) from veins of ear. The thighbone part of the house rabbit was subjected to local anesthesia with lidocaine chloride (2% Xylocalne E, AstraZeneca). The part was dissected until reaching periosteum, and the skin periosteum was peeled with dull knife to expose the portion around the joint of thighbone. An insertion hollow of 5.0 mm square was made under pouring of water, and the SPS gradient functional material was inserted in bone marrow cavity. The periosteum was subjected to intradermal suturing with absorptive suturing yarns (VICRIL 3-0, Ethicon), and the flap was sutured with nylon yarns (NESCOSUTURE, Nippon Shoji Co., Ltd.). As the control group, a gradient material comprising only AHAp was inserted, the periosteum was subjected to intradermal suturing with absorptive suturing yarns, and the flap was sutured with nylon yarns.

After breeding for 1, 3 months from the implantation of the gradient material, excess pentobarbital sodium was intravenously injected to kill the house rabbits, and the gradient material was extracted including the bone around the filled-up part. The extracted material was fixed with 10% neutral formalin solution for 2 weeks and used for preparation of tissue slice. The thighbones extracted with lapse of time were subjected to decalcification for 2 days with a rapid decalcifying solution (Decalcifying Solution A, WAKO) of Plank Rychlo formulation, then neutralized for a half day with 5% aqueous sodium sulfate solution, and embedded in paraffin by usual method to prepare a thin slice, which was double-stained with hematoxylin and eosine and used for histopathological retrieval. The animal experiment was conducted in accordance with the guideline for animal experiments.

4) Results

4)-1

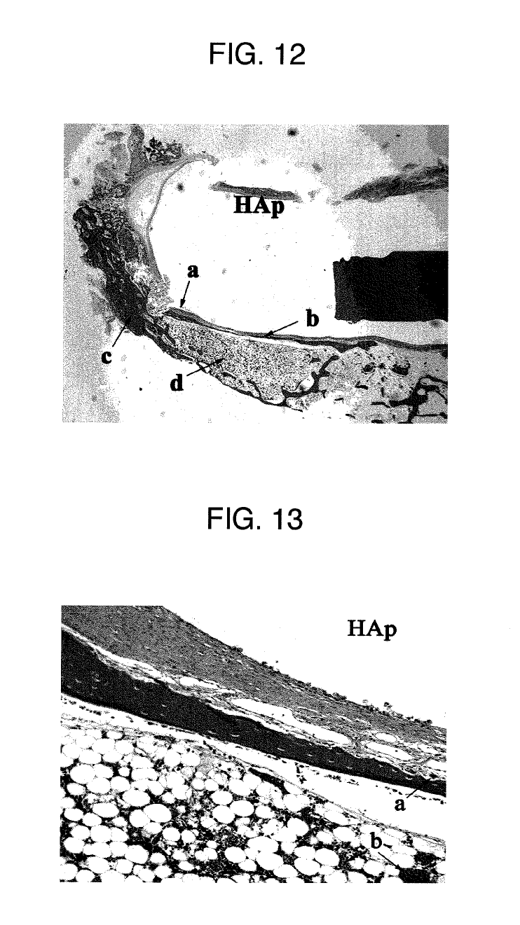

FIG. 12 shows a tissue reaction (3 months) in the control (comprising only HAp) which was enlarged in a small degree. In FIG. 12, the face contacting with HAp was encapsulated with fibrous bone (b) through a layer of fibrous connective tissue (a). This state shows that fibrous encapsulation occurs early and thereafter ossification occurred. It can be said that the state was not reactive, but stable since there were recognized lamellar bone (c) and fat cell (d) which was matured bone marrow tissue.

4)-2

FIG. 13 shows a tissue reaction (3 months) in the control (comprising only HAp) which was enlarged in a high degree. In highly enlarged drawing, osteoclasts (a) were seen, and reconstruction of bone occurred in some sites, which were a few. Presence of blood components was seen in osteoclasts, and there was formation of vascular system. No inflammatory cells were found.

4)-3

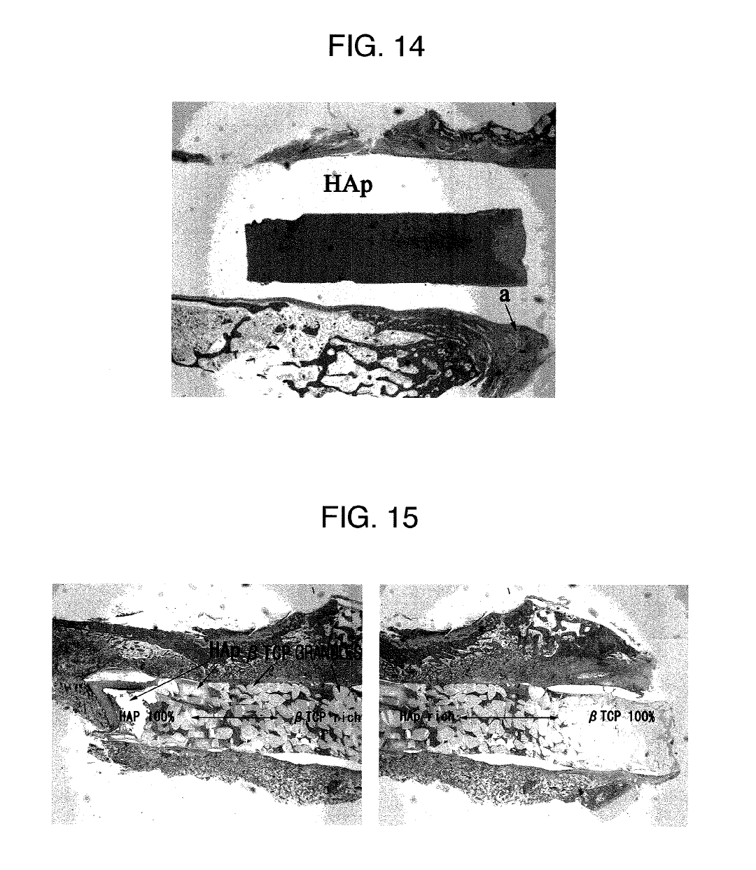

FIG. 14 shows a tissue reaction (3 months) in the control (comprising only HAp) which was enlarged in a small degree. In this FIG. 14, there was recognized formation of cartilage (a) around joint head. From this fact, it is recognized that ectopic ossification was not developed and tissue affinity was high.

4)-4

FIG. 15 shows a tissue reaction (3 months) in the test group (SPS gradient functional materials) which was enlarged in a small degree. In FIG. 15, gradient function was imparted to the test materials by SPS method so that the proportion of SHAp-100 gradually changed from 100% toward right side of 100% of β-TCP(L).

4)-5

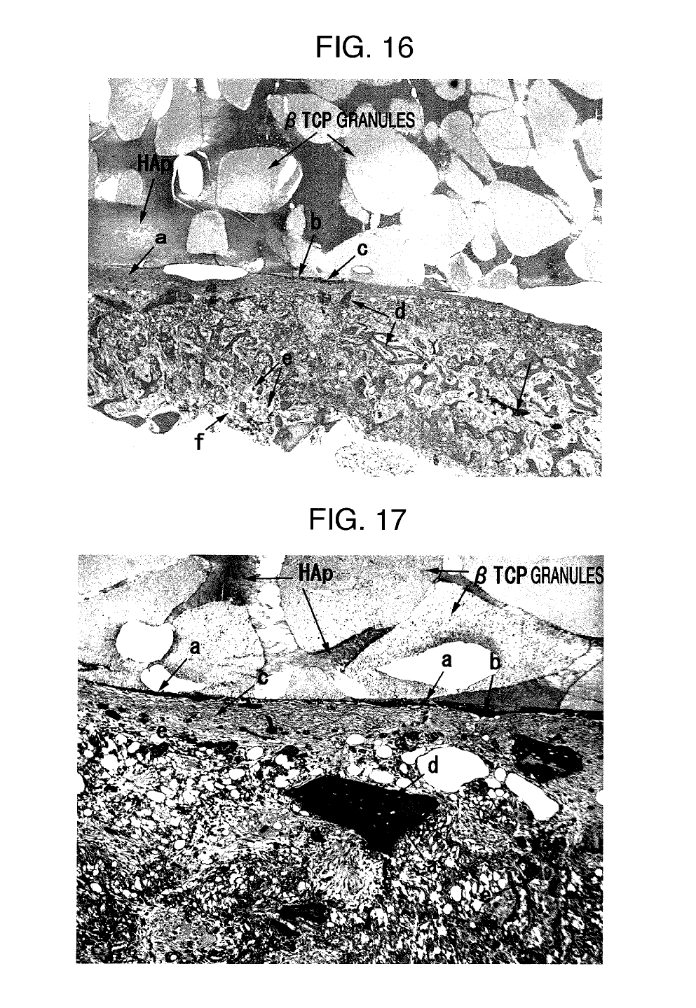

FIG. 16 shows the left side of tissue reaction (3 months) in the test group (SPS gradient functional materials) of FIG. 15, which was enlarged in a high degree. As can be seen from FIG. 16, the portion contacting with the test material was widely encapsulated with fibrous connective tissue (a), and the portion contacting with SHAp-100 was through fibrous connective tissue (a) while fibrous bone (b) and osteoblast (c) were seen in the portion which contacted with β-TCP(L) granules.

Outside the layer of fibrous connective tissue, there were bone marrow-derived tissue, and lamellar bone (d), osteoclast (e), fat cell (f) and the like were seen, and destruction and reconstruction of bone vigorously occurred. Particularly, since lamellar radius had irregular shape with many branches, it can be seen that reconstruction of bone vigorously occurred. There were recognized images of tissue higher in reactivity than that of the control.

4)-6

FIG. 17 shows the right side of tissue reaction (3 months) in the test group (SPS gradient functional materials) of FIG. 15 which was enlarged in a higher degree. As can be seen from FIG. 17, with increase of proportion of β-TCP(L) granules, proportions of the connective tissue (a) and fibrous bone (b) increased in the portion contacting with the material. Outside the portion, there was a layer of fibrous connective tissue (c). There were also recognized lamellar bone (d), osteoclast (e), and fat cell (f) which was considered to be derived from young bone marrow cells. It is considered that destruction and new bone reconstruction vigorously occurred since lamellar radius had irregular shape with many branches and the portion contacting with β-TCP(L) granules had particularly many fibrous bones.

4)-7

The above results show that in the case of HAp used as control, there were no inflammatory cell infiltration, but reactivity in new bone reconstruction was very low. On the other hand, in the case of using the gradient material, the tissues contacting with test material differed depending on the difference in compounding ratio of HAp and β-TCP(L) granules. As in the case of the control, the portion contacting with HAp was encapsulated with fibrous connective tissue, but in the portion contacting with β-TCP(L) granules where β-TCP was contained in a large amount, there were recognized images showing vigorous destruction and reconstruction of bones. That is, many osteoclasts appeared, and osteoblasts were present near the osteoclasts and they destructed the bone. However, since there were distributed lamellar bones having irregular shape, it can be seen that bone reconstruction also occurred. Fat cells which were considered to be derived from bone marrow cells were juvenile, and hence were considered to be in the state high in reactivity before maturation.

### INDUSTRIAL APPLICABILITY

As explained above, the layered gradient material for biological use according to the present invention is excellent in mechanical strength such as compressive strength, bending strength and Young's modulus, and further, is excellent in biological reactivity such that in the case where it is used as a bone substitute material, bone destruction and new bone reconstruction actively occur. Accordingly, it is extremely useful as a biological material such as a bone substitute material, artificial tooth root and dental cement.

### BRIEF DESCRIPTION OF DRAWINGS

[FIG. 1] shows construction of the layered body filled in die•punch made of high-strength graphite for producing the gradient material by SPS method in Example 1.

[FIG. 2] shows the die•punch made of high-strength graphite used for producing the gradient material by SPS method in Example 1.

[FIG. 3] shows an SEM photograph of the gradient material produced in Example 1.

[FIG. 4] shows the mold made of high-strength graphite used in producing the gradient material used for subjecting to compression test in Example 2.

[FIG. 5] shows construction of the layered body filled in die•punch made of high-strength graphite in producing the gradient material used for subjecting to compression test in Example 2.

[FIG. 6] shows the results of compression test in Example 2.

[FIG. 7] shows a mold made of high-strength graphite used in producing the gradient material used for measuring bending strength and Young's modulus in Example 2.

[FIG. 8] shows construction of the layered body filled in a die•punch made of high-strength graphite in producing the gradient material used for measuring bending strength and Young's modulus in Example 2.

[FIG. 9] shows the results of bending strength test in Example 2.

[FIG. 10] shows the results of measurement of Young's modulus in Example 2.

[FIG. 11] shows shape of specimen of the gradient material used in biological test in Example 3.

[FIG. 12] is a drawing enlarged in small degree which shows tissue reaction after 3 months of a material comprising only HAp used as a control in the biological test conducted in Example 3. In FIG. 12, a denotes fibrous connective tissue, b denotes fibrous bone, c denotes lamellar bone and d denotes fat cell.

[FIG. 13] is a drawing enlarged in high degree which shows tissue reaction after 3 months of a material comprising only HAp used as a control in the biological test conducted in Example 3. In FIG. 13, a denotes osteoclast and b denotes bone marrow cell.

[FIG. 14] is a drawing enlarged in small degree which shows tissue reaction after 3 months of a material comprising only HAp used as a control in the biological test conducted in Example 3. In FIG. 14, a denotes formation of cartilage.

[FIG. 15] is a drawing enlarged in small degree which shows tissue reaction after 3 months of the gradient material in the biological test conducted in Example 3.

[FIG. 16] is a drawing enlarged in high degree of the left side of the drawing enlarged in small degree of FIG. 15. In FIG. 16, a denotes fibrous connective tissue, b denotes fibrous bone, c denotes osteoblast, d denotes lamellar upper bone, e denotes osteoclast, and f denotes fat cell.

[FIG. 17] is a drawing enlarged in high degree of the right side of FIG. 15 enlarged in small degree.

1-13. (canceled)

14. A layered gradient material for biological use which comprises hydroxyapatite (HAp) having a particle size of 10 μm to 170 μm, and 13-phase tribasic calcium phosphate β-TCP) having a particle size of 590 μm to 1000 μm, the compounding ratio of HAp and β-TCP being changed stepwise or continuously, wherein the layered gradient material for biological use is obtained by stacking a plurality of layers in which the compounding ratio of HAp and β-TCP is changed stepwise or continuously, and then sintering the layered body by spark plasma sintering method (SPS method).

15. A layered gradient material for biological use according to claim 14 which is used as a bone substitute material.

16. A method for producing a layered gradient material for biological use which comprises stacking a plurality of layers composed of a mixture of HAp having a particle size of 10 μm to 170 μm and β-TCP having a particle size of 590 μm to 1000 μm in which the compounding ratio of HAp and β-TCP is changed stepwise or continuously, and then sintering the resulting layered body by spark plasma sintering method (SPS method).

17. A method for producing a layered gradient material for biological use according to claim 16, wherein a plurality of layers composed of a mixture of HAp and β-TCP in which the compounding ratio of HAp and β-TCP is changed stepwise or continuously are filled up in a frame and stacked in vertical direction to produce a layered body, and then the resulting layered body is sintered by SPS method.

18. A method for producing a layered gradient material for biological use according to claim 16 or 17.
